(19) United States
(12) Patent Application Publication
(10) Pub. No.: US 2019/0289227 A1
(43) Pub. Date: Sep. 19, 2019

(54) IMAGE PICKUP APPARATUS, IMAGE PICKUP SYSTEM, AND IMAGE PICKUP METHOD

(71) Applicant: OLYMPUS CORPORATION, Tokyo (JP)

(72) Inventor: Masahiro HIROSAWA, Tokyo (JP)

(73) Assignee: OLYMPUS CORPORATION, Tokyo (JP)

(21) Appl. No.: 16/253,901

(22) Filed: Jan. 22, 2019

(30) Foreign Application Priority Data
Mar. 16, 2018 (JP) 2018-049126

(51) Int. Cl. H04N 5/33; H04N 5/225; H04N 5/232; A61B 1/05; A61B 5/00

(52) U.S. Cl. CPC H04N 5/33; H04N 5/2253; A61B 5/0086; A61B 1/05; H04N 5/23229

(57) ABSTRACT

An image pickup system includes a thermography camera configured to pick up an image of an infrared light band, and a processor. The processor detects, from a difference in a temperature distribution between a thermography image for a subject obtained by image pickup of the subject using the thermography camera and a reference thermography image obtained by image pickup of the subject using the thermography camera, a change amount in the temperature distribution of the subject, detects a changed area of the subject where the detected change amount is a predetermined value or more, and generates a display image for displaying information about the detected changed area on a monitor.

This application claims benefit of Japanese Application No. 2018-49126 filed in Japan on Mar. 16, 2018, the contents of which are incorporated by this reference.

### BACKGROUND OF THE INVENTION

### 1. Field of the Invention

The present invention relates to an image pickup apparatus, an image pickup system, and an image pickup method using a thermography image.

### 2. Description of Related Art

Conventionally, an image pickup apparatus such as a camera has been used to observe and inspect a subject. Generally, in ordinary inspection using the image pickup apparatus, the image pickup apparatus displays an image of visible light on a monitor, and an inspector can judge a state of the subject by seeing a displayed image of the subject.

A thermography camera configured to display an image of infrared rays emitted by a subject on a monitor so that a temperature state of the subject can be visualized and displayed has also been widely used for various types of observation and inspection.

For example, Japanese Patent Application Laid-Open Publication No. 2001-286436 proposes an endoscope including a microbolometer element in which microbolometers are arranged in two dimensions and capable of measuring a temperature distribution of a subject. According to the proposed endoscope, an operator can grasp a temperature distribution within a body cavity.

### SUMMARY OF THE INVENTION

An image pickup apparatus according to an aspect of the present invention includes a thermography camera configured to pick up an image of an infrared light band, and a processor including a hardware, the processor being configured to: detect, from a difference in a temperature distribution between a thermography image for a subject obtained by image pickup of the subject using the thermography camera and a reference thermography image obtained by image pickup of the subject using the thermography camera, a change amount in the temperature distribution of the subject; detect a changed area of the subject where the detected change amount is a predetermined value or more; and generate a display image for displaying information about the detected changed area on a display device.

An image pickup system according to another aspect of the present invention includes an endoscope including an insertion section, a thermography camera provided in a distal end portion of the insertion section and configured to pick up an image of an infrared light band, and a processor including a hardware, the processor being configured to: detect, from a difference in a temperature distribution between a thermography image for a subject obtained by image pickup of the subject using the thermography camera and a reference thermography image obtained by image pickup of the subject using the thermography camera, a change amount in the temperature distribution of the subject; detect a changed area of the subject where the detected change amount is a predetermined value or more; and generate a display image for displaying information about the detected changed area on a display device.

An image pickup method according to still another aspect of the present invention includes picking up an image of a subject to acquire a thermography image for the subject using a thermography camera configured to pick up an image of an infrared light band, detecting, from a difference in a temperature distribution between the thermography image and a reference thermography image obtained by image pickup of the subject using the thermography camera, a change amount in the temperature distribution of the subject, detecting a changed area of the subject where the detected change amount is a predetermined value or more, and displaying information about the detected changed area on a display device.

### BRIEF DESCRIPTION OF THE DRAWINGS

FIG. 1 is a configuration diagram illustrating a configuration of an image pickup system according to a first embodiment of the present invention;

FIG. 2 is a block diagram illustrating a configuration of the image pickup system according to the first embodiment of the present invention;

FIG. 3 is a schematic view for describing a method for inspecting a turbine blade according to the first embodiment of the present invention;

FIG. 4 is a flowchart illustrating an example of flow of processing for acquiring a reference thermography image of a reference sample of the blade according to the first embodiment of the present invention;

FIG. 5 is a diagram for describing an arrangement relationship between the reference sample and a distal end portion when the reference thermography image of the reference sample of the blade is obtained according to the first embodiment of the present invention;

FIG. 6 is a diagram illustrating an example of the reference thermography image of the reference sample according to the first embodiment of the present invention;

FIG. 7 is a flowchart illustrating an example of flow of processing for inspecting each of blades by the image pickup system when each of blades of a rotor is inspected according to the first embodiment of the present invention;

FIG. 8 is a graph illustrating a temperature from a predetermined position P1 of a subject and a point Pd spaced apart from the position P1 by a distance d according to the first embodiment of the present invention;

FIG. 9 is a diagram illustrating an example of the blade including a crack according to the first embodiment of the present invention;

FIG. 10 is a diagram illustrating an example of a thermography image of the blade including the crack illustrated in FIG. 9;

FIG. 11 is a diagram illustrating an example of a difference image according to the first embodiment of the present invention;

FIG. 12 is a diagram illustrating an example of a blade including a thinned portion according to the first embodiment of the present invention;

FIG. 13 is a diagram illustrating an example of a thermography image of the blade including the thinned portion illustrated in FIG. 12;

FIG. 14 is a diagram illustrating an example of the difference image according to the first embodiment of the present invention;

FIG. 15 is a flowchart illustrating an example of flow of processing for inspecting each of blades by an image pickup system when each of blades of a rotor is inspected according to a modification 1 to the first embodiment of the present invention;

FIG. 16 is a flowchart illustrating an example of flow of processing for inspecting each of blades by an image pickup system when each of blades of a rotor is inspected according to a second embodiment of the present invention;

FIG. 17 is a flowchart illustrating an example of flow of processing for inspecting each of blades by an image pickup system when each of blades of a rotor is inspected according to a modification 2 to the second embodiment of the present invention;

FIG. 18 is a perspective view illustrating a configuration of an insertion section in an endoscope including a heating member according to a modification 3 to each of the embodiments and the modifications of the present invention;

FIG. 19 is a perspective view of a heating treatment instrument 61 having a bending habit in its distal end portion according to the modification 3 to each of the embodiments and the modifications of the present invention; and

FIG. 20 is a diagram for describing a bending mechanism of a heating treatment instrument according to the modification 3 to each of the embodiments and the modifications of the present invention.

### DETAILED DESCRIPTION OF PREFERRED EMBODIMENTS

Embodiments of the present invention will be described below with reference to the drawings.

### First Embodiment

### (System Configuration)

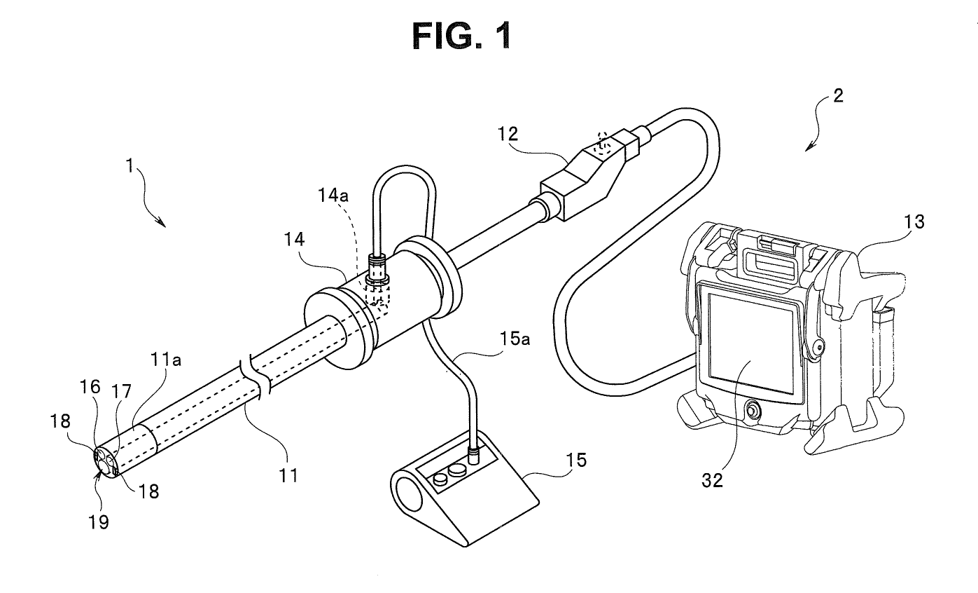

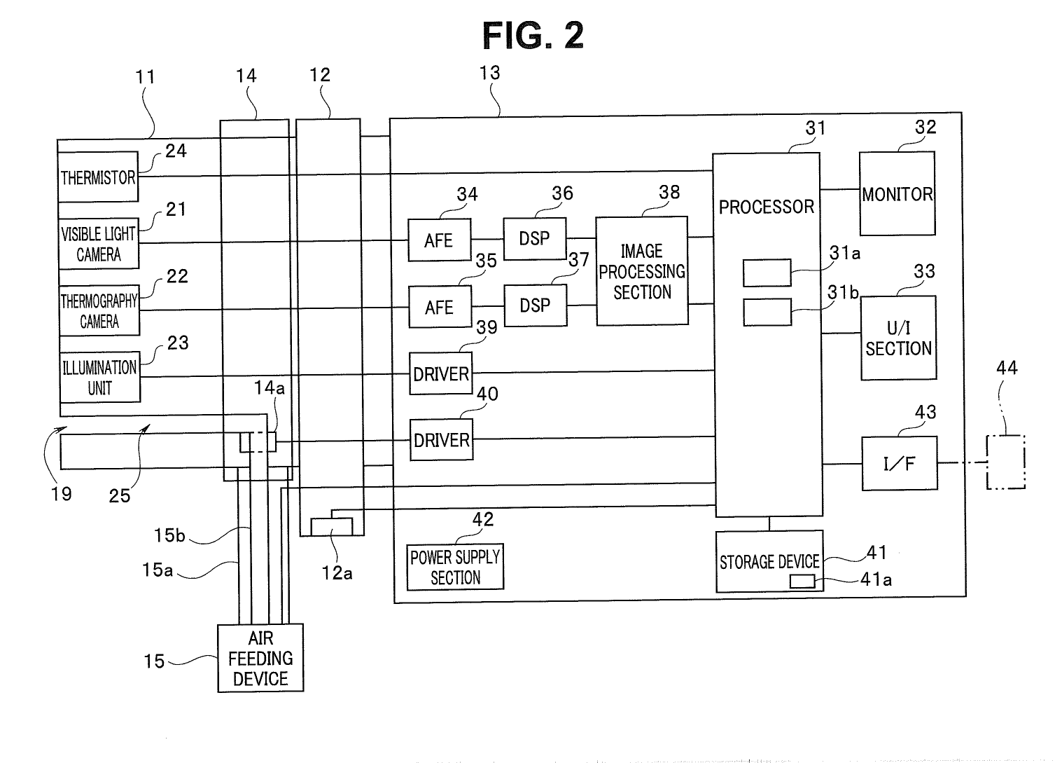

FIG. 1 is a configuration diagram illustrating a configuration of an image pickup system according to the present embodiment. FIG. 2 is a block diagram illustrating the configuration of the image pickup system.

An image pickup system 1 is an image pickup apparatus including an endoscope 2, and the endoscope 2 includes an elongated insertion section 11 configured to be inserted into a subject, an operation section 12, and a main body section 13. A heating device 14 configured to heat the subject is provided in the middle of the insertion section 11 in the endoscope 2. The heating device 14 is connected to an air feeding device 15 via a connection cable 15a. The heating device 14 is a device configured to heat a part of the subject by blowing hot air to the subject, as described below.

A distal end surface of a distal end portion 11a in the insertion section 11 is provided with a first observation window 16, a second observation window 17, two illumination windows 18, and an opening portion 19. The subject can be illuminated with illumination light emitted from each of the two illumination windows 18. The illumination light is white light.

Note that the insertion section 11 in the endoscope 2 will be described below as being elongated and rigid, the insertion section 11 may be a flexible insertion section having flexibility and further including a bending portion on the side of a proximal end of the distal end portion 11a.

A visible light camera 21 is disposed on the rear side of the first observation window 16. The visible light camera 21 includes an observation optical system and an image pickup device, and picks up an image of a visible light band to acquire a visible light image. The image pickup device in the visible light camera 21 is an image sensor configured to generate normal light image of the subject upon receiving light in the visible light band.

A thermography camera 22 is disposed on the rear side of the second observation window 17. The thermography camera 22 includes an observation optical system and an image pickup device, and picks up an image of an infrared light band. The image pickup device in the thermography camera 22 is an infrared image pickup device and an image sensor configured to generate a thermal image, i.e., a thermography image upon receiving infrared rays.

Two illumination units 23 each including a plurality of light emitting elements such as light emitting diodes (LEDs) are respectively provided on the rear side of the two illumination windows 18. Each of the illumination units 23 emits white light from the illumination window 18.

Furthermore, the distal end portion 11a is provided with a thermistor 24 as a temperature sensor. The thermistor 24 measures an ambient temperature of the distal end portion 11a, i.e., an environmental temperature of the subject.

The opening portion 19 communicates with an air feeding channel 25 formed within the insertion section 11. A proximal end portion of the air feeding channel 25 communicates with an air feeding device 15.

The operation section 12 includes an operation instrument 12a such as an operation button, and is operated by a user. An output signal of the operation instrument 12a is fed to a processor 31, described below, in the main body section 13.

The main body section 13 in the endoscope 2 includes the processor 31, a monitor 32, a user interface (U/I) section 33, two analog front ends 34 and 35, two digital signal processors (hereinafter referred to as DSPs) 36 and 37, an image processing section 38, two drivers 39 and 40, a large-capacity storage device 41, and a power supply section 42.

The processor 31 includes a central processing unit (hereinafter referred to as a CPU) 31a and a memory 31b. The memory 31b includes a ROM (read-only memory), a RAM (random access memory), and the like, and stores various types of processing programs.

Note that, although the processor 31 will be described herein as a device including the CPU 31a, the processor 31 may be at least one of a DSP (digital signal processor) and a GPU (graphics processing unit). Further, the entirety or a part of the processor 31 may be composed of a logical circuit or an analog circuit, and may include at least one of an ASIC (application specific integrated circuit) and a FPGA (field-programmable gate array), for example.

Further, one or two or more processors 31 may each include a temperature distribution change amount detection section, a changed area detection section, a display image generation section, or the like, described below.

The monitor 32 is a display device fixed to the main body section 13, for example, a liquid crystal panel.

The user interface (U/I) section 33 is a touch panel device provided in the monitor 32 or various types of buttons provided in the main body section 13, for example, and is operated by the user.

An image pickup signal from the visible light camera 21 is inputted to the analog front end 34, and is outputted after being converted into a digital signal. An output signal of the analog front end 34 is inputted to the DSP 36, is subjected to various types of image processing, and is outputted to the image processing section 38.

The image pickup signal from the thermography camera 22 is inputted to the analog front end 35, and is outputted after being converted into a digital signal. An output signal of the analog front end 35 is inputted to the DSP 37, is subjected to various types of correction processing, and is outputted to the image processing section 38.

Respective image signals from the DSPs 36 and 37 are each subjected to various types of image processing in the image processing section 38. The image processing section 38 generates a visible light image and a thermography image to be displayed on the monitor 32 by various types of image processing, and outputs the generated images to the processor 31.

The illumination unit 23 is driven by the driver 39, to emit illumination light. The driver 39 is connected to the processor 31, to operate in response to a control signal from the processor 31.

An output signal of the thermistor 24 is inputted to the processor 31.

The user interface (U/I) section 33 and the operation instrument 12a are connected to the processor 31. The user can issue various types of instructions to the processor 31 by operating the user interface (U/I) section 33 or the operation instrument 12a.

The main body section 13 includes an interface (I/F) 43, and the processor 31 is connected to a turning device 44, described below, by the interface 43.

The CPU 31a in the processor 31 reads out and executes a software program stored in the memory 31b in response to the instruction from the user, to exhibit various types of functions of the image pickup system 1. Examples of the various types of functions include display, recording, data input, and the like of an endoscope image of visible light, display, recording, data input, and the like of a thermography image, and further detection of presence or absence of an abnormal area of the subject which is not seen or is not easily recognized by the user, described below.

The heating device 14 includes a heat generation unit 14a including a heating element. Examples of the heating element in the heat generation unit 14a include a Peltier element, a heater element, a microwave generator, and the like. The heat generation unit 14a is provided to closely adhere to an outer peripheral portion of an air feeding tube 15b, described below, to heat air from the air feeding device 15.

The air feeding device 15 is connected to the heating device 14 via a connection cable 15a. The air feeding tube 15b is inserted into the connection cable 15a. The air feeding device 15 can feed air into the air feeding channel 25 in the insertion section 11 by the air feeding tube 15b.

The air feeding device 15 includes a pump (not illustrated) and a flowmeter (not illustrated). A proximal end of the air feeding tube 15b is connected to the pump. The air feeding device 15 can feed a predetermined feeding amount of air into the air feeding tube 15b by the pump based on a measurement value of the flowmeter.

A distal end of the air feeding tube 15b communicates with the air feeding channel 25 within the heating device 14. Accordingly, the air from the air feeding device 15 is heated by the heat generation unit 14a, and is discharged from the opening portion 19 in the distal end portion 11a through the air feeding channel 25.

The storage device 41 is connected to the processor 31, and inspection data such as a visible light image as an endoscope image is recorded in the storage device 41. The storage device 41 includes a storage region 41a storing a reference thermography image TIREF, described below, and table data TBL of correction data, described below.

The storage device 41 is configured herein as a volatile or nonvolatile memory. For example, the storage device 41 includes at least one of a RAM (random access memory), a DRAM (dynamic random access memory), an SRAM (static random access memory), an EPROM (erasable programmable read only memory), an EEPROM (electrically erasable programmable read-only memory), a flash memory, and a hard disk drive.

The power supply section 42 supplies power to each of the sections in the image pickup system 1. The power supply section 42 includes a secondary battery, for example.

The user as an inspector can perform observation and inspection within the subject by inserting the insertion section 11 into the subject, operating the operation instrument 12a or the like to display an endoscope image of an inspection site picked up by the visible light camera 21 on the monitor 32.

The user can confirm a temperature distribution within the subject by operating the operation instrument 12a or the like to display a thermal image of an inspection site picked up by the thermography camera 22 on the monitor 32.

Furthermore, the user can detect presence or absence of an abnormal area such as a fine crack or a thinned portion of the subject which is not seen or is not easily recognized by the user in the visible light image, by heating a part of the subject using the image pickup system 1, from a change in a heat distribution after the heating. The crack or the like is a changed area which has occurred in a part of the subject as a result of long-term use of the subject. In the inspection, it is confirmed whether or not a changed area exists and whether or not no problem occurs even if the changed area exists, for example.

The user can also record a visible light image, a thermography image, abnormal area information, or the like as inspection result information in the storage device 41.

A case where the image pickup system 1 inspects a plurality of turbine blades in an electric generator will be described below. The subject in the present embodiment is a plurality of turbine blades having an identical shape.

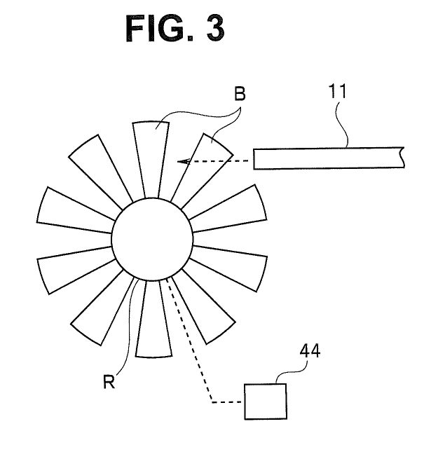

FIG. 3 is a schematic view for describing a method for inspecting the turbine blades. A turbine includes a rotor R that rotates around a rotation axis of the turbine. The plurality of turbine blades (hereinafter referred to as blades) B are equally spaced around an axis of the rotor R.

The turning device 44 is connected to a gear box (not illustrated) of the turbine so that the plurality of blades B can be rotated around a rotation axis of the rotor R.

The turning device 44 is connected to the main body section 13 in the image pickup system 1 via a cable, to rotate the rotor R in response to an instruction from the main body section 13. The turning device 44 is controlled by the processor 31.

Note that the turning device 44 is not connected to the endoscope 2 but may be controlled by another control device so that the rotor R is rotated.

A casing of the turbine includes an access port as a through hole at a predetermined position. The insertion section 11 can be inserted into the casing via the access port. Accordingly, the endoscope 2 is an elongated borescope having such a diameter and a length that the blade B within the turbine can be observed.

The distal end portion 11a in the insertion section 11 is fixed while being directed toward the subject so that the user can observe and inspect each of the blades B as the subject by acquiring a visible light image and a thermography image of the blade B.

### (Function)

An operation of the image pickup system 1 will be described below. An example in which the above-described turbine blade is inspected is used to describe the operation of the image pickup system 1.

Inspection of a plurality of blades having an identical shape, respectively, as subjects will be described below as an example. Needless to say, however, the image pickup system 1 can be used to inspect not only a plurality of subjects having an identical shape but also a single subject. If the subject is a casting, for example, the image pickup system 1 can detect presence or absence of an abnormal area by acquiring a thermography image of the manufactured casting.

If the subject is a piping, the piping is previously divided into a plurality of regions so that the image pickup system 1 can detect presence or absence of an abnormal area by acquiring a thermography image of each of the regions.

First, acquisition and recording of a thermography image of a reference sample stored in the storage region 41a in the storage device 41 in the main body section 13 will be described.

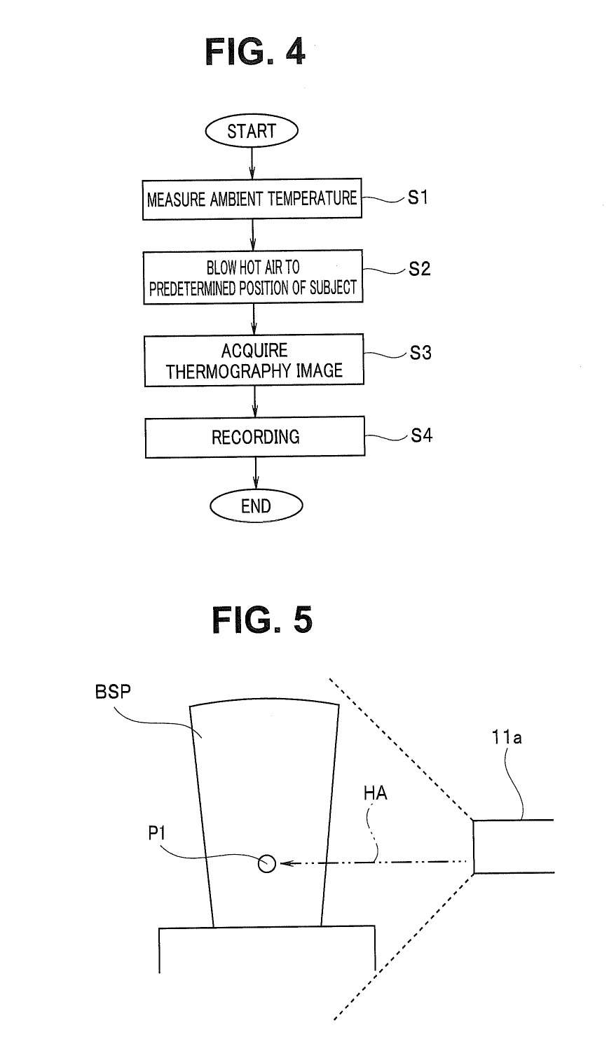

FIG. 4 is a flowchart illustrating an example of flow of processing for acquiring a reference thermography image of a reference sample BSP of a blade B. A program for the processing illustrated in FIG. 4 is stored in the ROM in the memory 31b, is read out in response to an instruction from the user, and is executed by the CPU 31a.

The user as an inspector prepares the reference sample BSP of the blade B as the subject. The reference sample BSP is a normal blade including no abnormal area such as a crack or a chip, and is the same in shape, size, and material as the blade B as the subject.

When the user fixes the reference sample BSP to a predetermined instrument, and operates the user interface (U/I) section 33 with the distal end portion 11a in the insertion section 11 positioned with respect to the reference sample BSP at the same position as when the blade B is inspected, to input a predetermined command, the processor 31 performs the processing illustrated in FIG. 4.

The processor 31 measures an ambient temperature of the subject (step (hereinafter abbreviated as S) 1). The processor 31 calculates an ambient temperature of the reference sample BSP from an output signal of the thermistor 24.

Then, the processor 31 blows air heated by the heat generation unit 14a, i.e., hot air to a predetermined position P1 of the reference sample BSP for a predetermined time period T1 (S2). The hot air is ejected from the opening portion 19 in the distal end portion 11a, and is applied to the predetermined position P1 of the blade B for the predetermined time period T1.

FIG. 5 is a diagram for describing an arrangement relationship between the reference sample BSP and the distal end portion 11a when the reference thermography image of the reference sample BSP of the blade B is obtained. In FIG. 5, a dotted line indicates a range included in the visible light image and the thermography image.

The distal end portion 11a is arranged at the same position and in the same posture as when hot air is applied to a predetermined position of each of the blades B of the rotor R when the blade B is actually inspected. Accordingly, a distance between the distal end portion 11a and the reference sample BSP is equal to a distance between the distal end portion 11a and each of the blades B of the rotor R when the blade B is actually inspected.

In FIG. 5, hot air HA is blown toward the predetermined position P1 of the reference sample BSP. The hot air HA is ejected from the opening portion 19, to heat the predetermined position P1 of the reference sample BSP. The predetermined position P1 is a position closer to a proximal end on a longitudinal axis of the blade B.

A predetermined time period T1 during which the hot air HA is ejected from the opening portion 19 is ten seconds, for example. The predetermined time period T1 is equal to a time period during which the hot air HA is ejected when each of the blades B of the rotor R is actually inspected. A temperature of the hot air HA is also equal to a temperature of the hot air HA when each of the blades B of the rotor R is actually inspected.

When blowoff of the hot air is finished, the processor 31 acquires a thermography image (S3). That is, the thermography image TI acquired in S3 is a reference thermography image TIREF obtained by image pickup using the thermography camera 22 after the heating device 14 heats a part of a sample as a reference of a subject for the predetermined time period T1. The reference thermography image TIREF is an image obtained by image pickup of the sample as the reference of the subject.

When the predetermined position P1 of the reference sample BSP is heated by the hot air HA at a predetermined temperature, heat is conducted to a portion around the predetermined position P1 of the reference sample BSP. The thermography image acquired in S3 is an image representing a heat distribution of the reference sample BSP as a result of heat conduction.

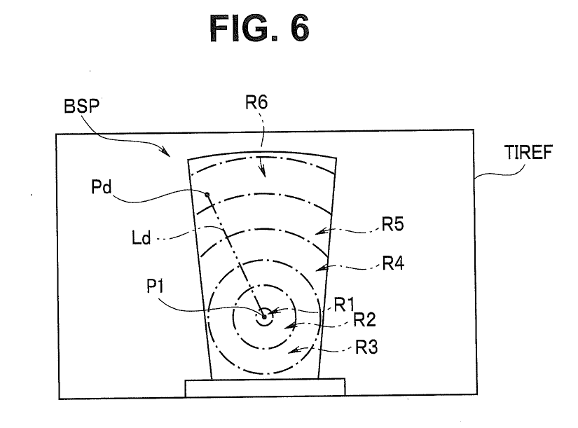

FIG. 6 is a diagram illustrating an example of the reference thermography image TIREF of the reference sample BSP.

The reference thermography image TIREF of the reference sample BSP is highest in temperature at a predetermined position P1 of the reference sample BSP and gradually decreases in temperature radially around the predetermined position P1, as illustrated in FIG. 6. A temperature of a region R1 including the predetermined position P1 is highest, and heat is conducted concentrically from the predetermined position P1 around the region R1. Accordingly, a thermography image including a plurality of regions, regions R2, R3, R4, R5, and R6 which decrease in temperature in this order from the predetermined position P1 toward an outer diameter direction is obtained as the reference thermography image TIREF. The reference thermography image TIREF illustrated in FIG. 6 has a concentric heat distribution.

After S3, the processor 31 stores image data of the obtained reference thermography image TIREF in the storage region 41a in the storage device 41 (S4). Accordingly, the storage region 41a constitutes a storage section configured to store the reference thermography image TIREF.

The reference thermography image TIREF is acquired in a manner described above, and is registered in the storage device 41.

Then, an operation of the image pickup system 1 performed when the turbine blade is actually inspected will be described below. An operation performed when presence or absence of an abnormal area such as a fine crack or a thinned portion of the subject which is not seen or is not easily recognized by the user in the visible light image is detected will be described.

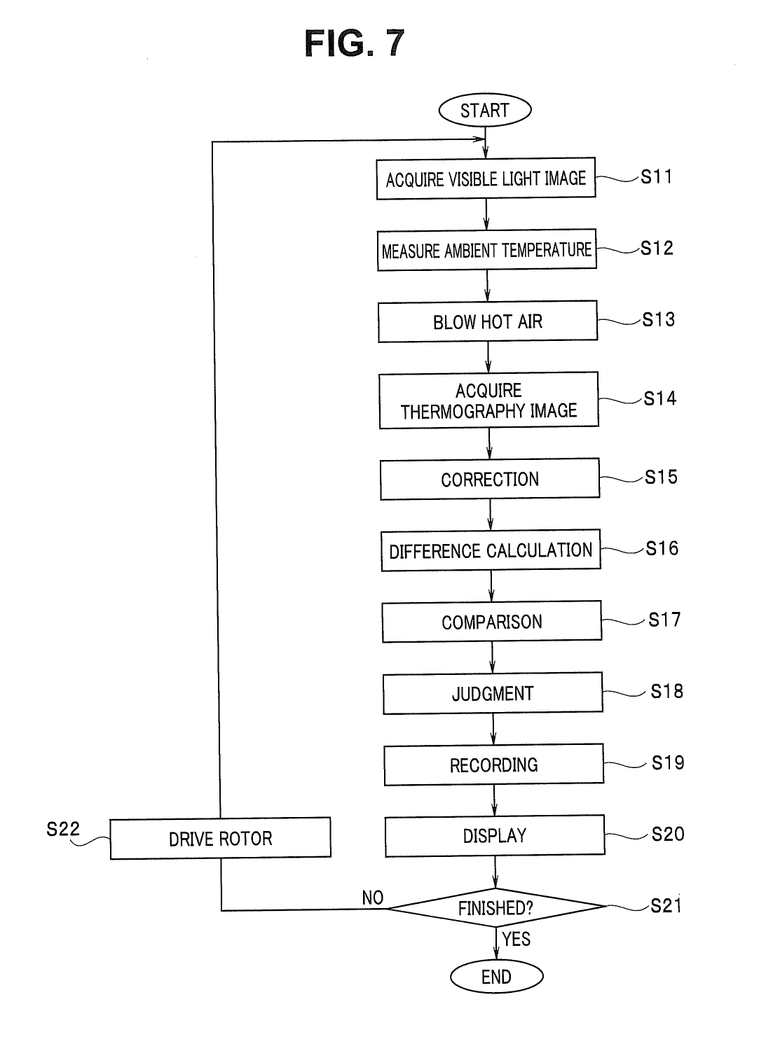

FIG. 7 is a flowchart illustrating an example of flow of processing for inspecting each of the blades B by the image pickup system when each of the blades B of the rotor R is inspected. A program for the processing illustrated in FIG. 7 is stored in the ROM in the memory 31b, is read out in response to an instruction from the user, and is executed by the CPU 31a.

As described above, when the inspection is performed, the user inserts the insertion section 11 from an access port of the rotor R, and sequentially picks up respective images of the blades B of the rotor R. The processor 31 controls, when the one blade B has been inspected after the image of the blade B is picked up, the turning device 44 such that the blade B adjacent to the blade B which has been inspected enters an image pickup range of the distal end portion 11a in the insertion section 11, and inspects the blade B adjacent to the blade B. When such processing is repeated, all the blades B of the rotor R are inspected.

As inspection result information, information about an abnormal area, as described below, is also recorded in the storage device 41 in addition to a visible light image by visible light and a thermography image by infrared rays. The abnormal area is an area where a temperature distribution of the subject changes, as described below.

First, when the user sets the distal end portion 11a in the insertion section 11 to a predetermined position with respect to the first blade B and operates the user interface (U/I) section 33 or the operation instrument 12a, the processor 31 acquires a visible light image of the blade B (S11). That is, the processor 31 acquires a visible light image of a subject by reflected light from the subject which has passed through the first observation window 16, i.e., an endoscope image by normal light.

Subsequently to S11, the processor 31 measures an ambient temperature of the blade B (S12). The measurement of the ambient temperature of the blade B is calculated from an output signal of the thermistor 24.

Subsequently to S12, the processor 31 blows hot air HA to the blade B (S13). Thus, the predetermined position P1 of the blade B is heated. More specifically, the processor 31 drives the pump in the air feeding device 15 with the heat generation unit 14a in the heating device 14 generating heat such that the hot air HA is applied to the predetermined position P1 of the blade B for the predetermined time period T1.

At this time, the heat generation unit 14a is driven to generate heat such that the hot air HA at the same temperature as the temperature of the hot air HA blown to the reference sample BSP in S2 illustrated in FIG. 5 is blown to the blade B.

When the blowing of the hot air HA from the opening portion 19 is finished, the processor 31 acquires a thermography image TI (S14). The predetermined position P1 of the blade B is heated for the predetermined time period T1 by the hot air HA, and the heat is propagated to a periphery of the predetermined position P1 of the blade B. That is, the thermography image TI acquired in S14 is an image obtained by heating a part of the subject by the heating device 14 for the predetermined time period T1, followed by image pickup using the thermography camera 22. Note that, when the ambient temperature of the blade B varies, the thermography image TI obtained in S14 also varies.

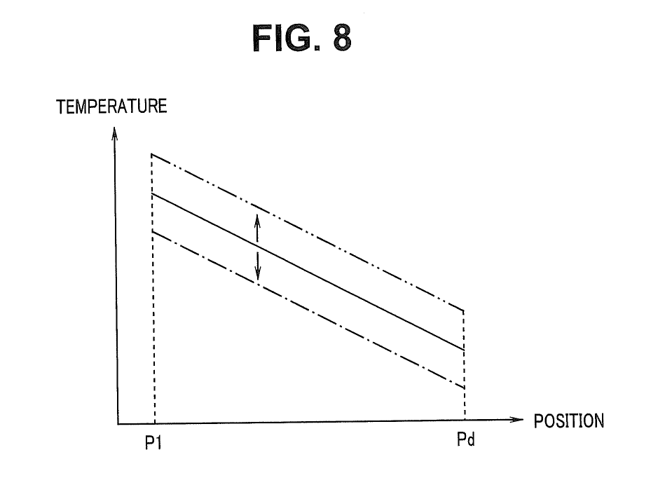

FIG. 8 is a graph illustrating a temperature from the predetermined position P1 to a point Pd spaced apart from the predetermined position P1 by a distance d. FIG. 8 schematically illustrates a temperature along a virtual line Ld connecting the predetermined position P1 illustrated in FIG. 6 and the point Pd spaced apart from the predetermined position P1 by the distance d.

When an ambient temperature of the blade B differs from an ambient temperature of a reference sample BSP at the time when the reference thermography image TIREF is obtained, a heat distribution varies. If an ambient temperature Tm of the blade B is the same as an ambient temperature Tr of the reference sample BSP at the time when the reference thermography image TIREF is obtained, a temperature at each of points on the virtual line Ld in the blade B substantially matches a temperature at the point on the virtual line Ld in the reference sample BSP. In FIG. 8, a temperature distribution is indicated by a solid line.

When the ambient temperature Tm of the blade B is lower than the ambient temperature Tr of the reference sample BSP, a temperature at each of the points on the virtual line Ld in the blade B becomes lower than a temperature at the point on the virtual line Ld in the reference sample BSP. Accordingly, the graph illustrated in FIG. 8 becomes a graph having a temperature distribution indicated by a one-dot and dash line.

When the ambient temperature Tm of the blade B is higher than the ambient temperature Tr of the reference sample BSP, a temperature at each of the points on the virtual line Ld in the blade B becomes higher than a temperature at the point on the virtual line Ld in the reference sample BSP. Accordingly, the graph illustrated in FIG. 8 becomes a graph having a temperature distribution indicated by a two-dot and dash line.

Correction coefficient data corresponding to a difference between the ambient temperature Tm of the blade B as the subject and the ambient temperature Tr, at the time when the reference thermography image TIREF has been obtained, of the reference sample BSP is previously held as table data TBL in the storage region 41a in the storage device 41.

The correction coefficient data may be generated by being experimentally found, for example.

After S14, the processor 31 reads out the correction coefficient data from the table data TBL in the storage region 41a based on the ambient temperature obtained in S12, to correct each of pixel values of the thermography image TI obtained in S14 (S15).

The processor 31 performs difference calculation between the corrected thermography image TI and the reference thermography image TIREF (S16). The processor 31 reads out the reference thermography image TIREF from the storage device 41, stores the read reference thermography image TIREF in the RAM in the memory 31b, uses the reference thermography image TIREF when S16 is performed.

The difference calculation is performed by taking a difference between respective pixel values for each pixel of the corrected thermography image TI and the reference thermography image TIREF, to generate a difference image. The difference image is an image having a difference value between respective pixel values of two corresponding pixels at the same position of two images.

That is, a process in S16 constitutes a temperature distribution change amount detection section configured to detect a change amount in a temperature distribution of a subject from a difference in temperature distribution between the thermography image TI for the subject obtained by image pickup of the subject using the thermography camera 22 and the reference thermography image TIREF obtained by image pickup using the thermography camera 22. In S16, a change amount is detected based on the respective pixel values for each pixel of the thermography image TI and the reference thermography image TIREF.

Accordingly, if the corrected thermography image TI has the same heat distribution as the heat distribution of the reference thermography image TIREF illustrated in FIG. 6, each of difference values becomes zero. Thus, the difference image obtained in S16 becomes a black image.

Note that a position, with respect to the blade B, of the distal end portion 11a may not be easily made to accurately match a position, with respect to the reference sample BSP, of the distal end portion 11a at the time when the reference thermography image TIREF is acquired. Accordingly, difference calculation may be performed after pattern matching between the reference thermography image TIREF and the thermography image TI of the blade B is performed to adjust a shift of the thermography image TI with respect to the reference thermography image TIREF.

The processor 31 compares a pixel value of each of pixels composing the obtained difference image with a predetermined threshold value TH (S17). A process in step S17 constitutes a changed area detection section configured to detect a changed area of the subject where the change amount detected in S16 is a predetermined value or more.

After S17, the processor 31 judges presence or absence of an abnormal area (S18).

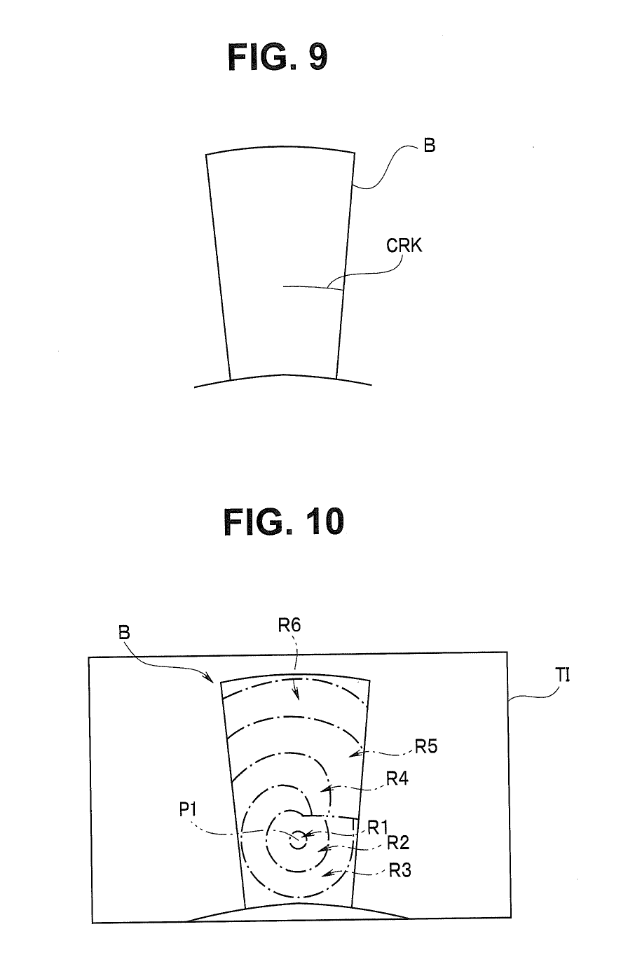

FIG. 9 is a diagram illustrating an example of a blade B including a crack.

A thin crack CRK occurs in a part of the blade B illustrated in FIG. 9. If the crack CRK can be seen on a visible light image, the user can find out that the crack CRK exists. However, if the crack CRK is narrow and thin, the user may be unable to find out that the crack CRK exists by seeing the visible light image.

However, if the crack CRK exists, heat is not easily transmitted in an area where the crack CRK exists. Therefore, in S17, a difference image is generated, and the processor 31 judges presence or absence of an abnormal area depending on whether or not each of pixel values of the difference image is a predetermined threshold value TH or more.

FIG. 10 is a diagram illustrating an example of a thermography image TI of the blade B including the crack CRK illustrated in FIG. 9. As illustrated in FIG. 10, heat applied to a predetermined position P1 is transmitted in all outer diameter directions from the predetermined position P1. Although heat is concentrically transmitted from the predetermined position P1 to the crack CRK, the crack CRK causes the transmission of the heat to be inhibited or prevented. As a result, the thermography image TI having a heat distribution, as illustrated in FIG. 10, is obtained in S14.

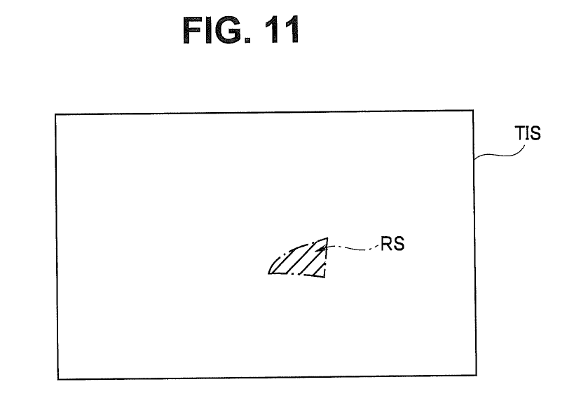

A difference image TIS between the thermography image TI illustrated in FIG. 10 and the reference thermography image TIREF illustrated in FIG. 6 becomes an image as illustrated in FIG. 11, for example. FIG. 11 is a diagram illustrating an example of the difference image.

In FIG. 11, a region RS indicated by an oblique line represents a region of a pixel having a pixel value which is a threshold value TH or more in the difference image. The region RS occurs due to existence of the crack CRK. The region RS is an image representing a heat distribution, although indicated by the oblique line in FIG. 11.

If the crack CRK does not exist, the region RS of the pixel having the pixel value which is the predetermined threshold value TH or more does not exist in the difference image.

Accordingly, since the region RS exists by the comparison in S17, the processor 31 can judge that the blade B includes an abnormal area (S18).

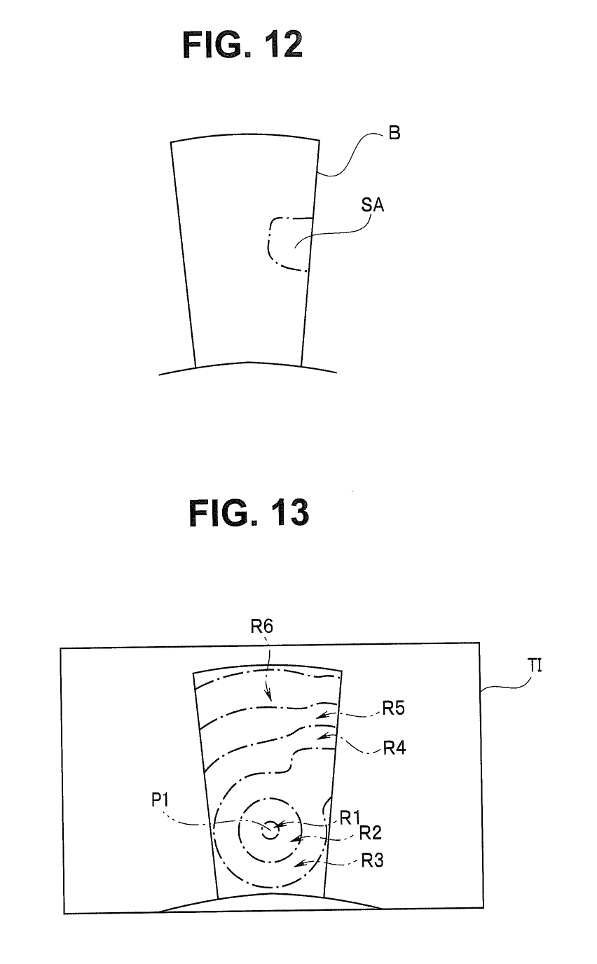

Although examples of the abnormal area include various abnormal areas, FIG. 12 is a diagram illustrating an example of a blade B including a thinned portion. The blade B is a bent and plate-shaped member. However, even if a thinned portion, i.e., a thinned portion SA exists in the blade B on the right side of a central portion, as illustrated in FIG. 12, for example, the existence of the thinned portion SA may not be able to be confirmed with naked eyes of the user in a normal endoscope image.

When processes in S13 to S17, described above, are performed for the blade B including the thinned portion SA, the existence of the thinned portion SA can be judged.

FIG. 13 is a diagram illustrating an example of the thermography image TI of the blade B including the thinned portion SA illustrated in FIG. 12. As illustrated in FIG. 13, heat applied to a predetermined position P1 is transmitted in all outer diameter directions from the predetermined position P1. Although heat is concentrically equally transmitted from the predetermined position P1 to the thinned portion SA, heat is fast transmitted in a portion of the thinned portion SA. As a result, a thermography image TI having a heat distribution, as illustrated in FIG. 13, is obtained in S14.

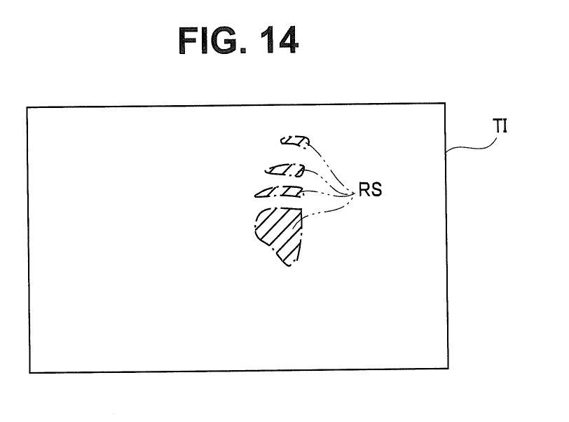

A difference image between the thermography image TI illustrated in FIG. 13 and the reference thermography image TIREF illustrated in FIG. 6 becomes an image as illustrated in FIG. 14, for example. FIG. 14 is a diagram illustrating an example of the difference image.

In FIG. 14, a region RS indicated by an oblique line represents a region of a pixel having a pixel value which is a threshold value TH or more in the difference image. The region RS occurs due to existence of the thinned portion SA. The region RS illustrated in FIG. 14 is also an image representing a heat distribution, although indicated by the oblique line.

If the thinned portion SA does not exist, the region RS of the pixel having the pixel value which is the predetermined threshold value TH or more does not exist in the difference image.

Accordingly, since the region RS exists, the processor 31 judges that an abnormal area exists or an abnormal area may exist in the blade B (S18).

In S18, after the presence or absence of the abnormal area is judged, the processor 31 records inspection result information about the inspected blade B in the storage device 41 (S19).

In the recording processing in S19, an endoscope image of visible light, a thermography image, a judgment result, and a difference image at the time when the abnormal area has been found out are recorded in a memory as the inspection result information about the inspected blade. The judgment result information includes information such as a flag representing presence or absence of an abnormal area or presence or absence of a possibility of the abnormal area.

The processor 31 displays, if an abnormal area has not been found out as a result of the judgment, a message indicating that no abnormal area exists on the monitor 32, and displays, when an abnormal area has been found out, a message indicating that an abnormal area exists, a difference image, and the like on the monitor 32 (S20).

For example, when the abnormal area has been found out, display processing for displaying an image obtained by superimposing a difference image on a visible light image on the monitor so that the abnormal area is found for the user is performed. That is, as information about a changed area of a detected temperature distribution, a difference image between the thermography image TI and the reference thermography image TIREF is displayed. The user can simply grasp a position or a region of the abnormal area on the visible light image.

Alternatively, a predetermined mark representing an abnormal area, e.g., a circle mark or an arrow may be displayed on the visible light image.

Accordingly, a process in S20 constitutes a display image generation section configured to generate a display image for displaying information about the changed area of the subject detected in S17 on the monitor 32. In S20, information about the changed area of the subject, e.g., a mark is superimposed on the visible light image obtained by image pickup using the visible light camera 21.

Note that a predetermined mark or the like may be displayed while being superimposed as the information about the changed area on the thermography image.

The processor 31 judges whether or not image acquisition, judgment processing, or the like has been finished for all the blades B of the rotor R, i.e., whether or not image acquisition or the like for the last blade B has been finished (S21).

When the image acquisition or the like for all the blades B has been finished (YES in S21), the processing ends.

When the image acquisition or the like for all the blades B has not been finished (NO in S21), the processor 31 rotates the rotor R by a predetermined angle (S22). After the adjacent blade B has reached a position where an image of the blade B can be picked up using the endoscope in S22, the processor 31 performs processes in S11 and subsequent steps.

As described above, according to the above-described embodiment, the image pickup apparatus, the image pickup system, and the image pickup method capable of detecting the area which cannot be found out or is not easily found out in the visible light image can be implemented.

Note that, although the reference thermography image is an image obtained by heating the reference sample BSP of the blade B, like at the time of inspection, a thermography image obtained at the time of past inspection for the blade B may be used as a reference thermography image. For example, for the blade B which has been judged to be normal in previous inspection, a current thermography image and a previous thermography image may be compared with each other.

A modification to the above-described first embodiment will be described below.

### Modification 1

In the above-described embodiment, the reference thermography image TIREF is generated using the reference sample BSP of the blade B to be inspected, and is previously stored in the storage device 41, and the thermography image for each of the blades and the reference thermography image TIREF are compared with each other. On the other hand, in the modification 1, a reference thermography image TIREF is not previously acquired but is determined from a plurality of thermography images of a plurality of blades that have been inspected.

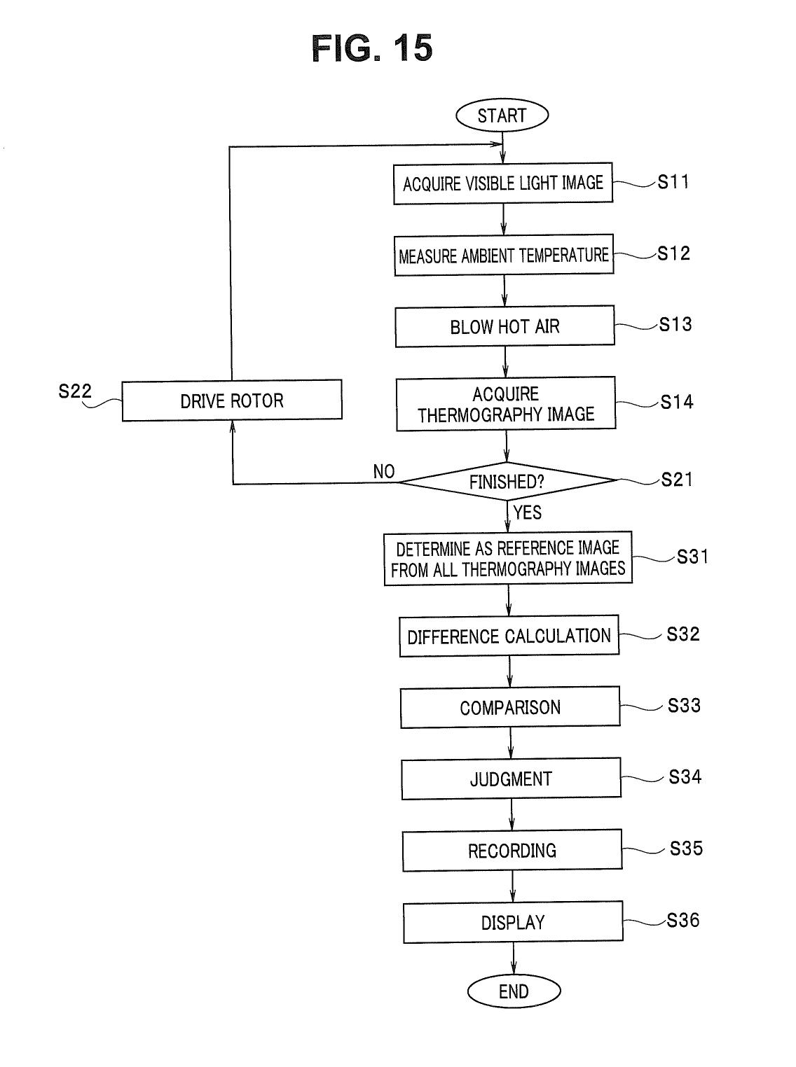

FIG. 15 is a flowchart illustrating an example of flow of processing for inspecting each of blades B by an image pickup system 1 when each of blades B of a rotor R is inspected according to the modification 1. Note that in steps illustrated in FIG. 15, the same processes as the processes illustrated in FIG. 7 are respectively assigned the same step numbers and are briefly described.

A procedure performed when inspection is started is the same as the procedure performed in the above-described embodiment.

First, when a user sets the first blade B to a predetermined position with respect to a distal end portion 11a in an insertion section 11 and operates a user interface (U/I) section 33 or an operation instrument 12a, a processor 31 performs processing illustrated in FIG. 15 to first acquire a visible light image of the blade B (S11).

After S11, the processor 31 measures an ambient temperature of the blade B (S12).

Subsequently to S12, the processor 31 blows hot air HA to the blade B (S13).

Subsequently to S13, the processor 31 acquires a thermography image TI (S14).

After S14, the processor 31 judges whether or not processes in S11 to S14 have been finished for all the blades B of the rotor R (S21).

When image acquisition for all the blades B has not been finished (NO in S21), the processor 31 performs a process in S22, and the processing then returns to S11.

When the processes in S11 to S14 have been finished for all the blades B (YES in S21), the processor 31 determines the reference thermography image TIREF from all respective thermography images TI of all the blades B (S31).

That is, a process in S31 constitutes a reference image determination section configured to determine, when subjects exist, the reference thermography image TIREF based on the plurality of thermography images TI obtained by image pickup of a plurality of subjects using a thermography camera 22.

Various methods are used to determine the reference thermography image TIREF.

For example, an average image of all the thermography images TI is generated, and is set as the reference thermography image TIREF. In this case, the average image is preferably generated from the plurality of thermography images excluding the thermography image having a pixel value greatly different from each of pixel values of the average image.

Alternatively, when a sum of all pixel values of a difference image between the one thermography image TI and the above-described average image is smallest, the thermography image TI may be extracted and set the thermography image TI as the reference thermography image TIREF.

After S31, the processor 31 performs difference calculation between each of the thermography images TI acquired in S14 and the reference thermography image TIREF determined in S31 (S32). The difference calculation in S32 is performed for all the thermography images TI in S32, although similar to the process in S16. A process in S32 constitutes a temperature distribution change amount detection section configured to detect a change amount in a temperature distribution of the subject.

After S32, the processor 31 compares a pixel value of each of pixels composing each of the obtained difference images with a predetermined threshold value TH (S33). The comparison calculation in S33 is performed for all the thermography images in S33, although similar to the process in S17. A process in S33 constitutes a changed area detection section configured to detect a changed area of the subject.

After S33, the processor 31 judges presence or absence of an abnormal area (S34). The judgment processing in S34 is performed for all comparison calculation results of all the thermography images in S34, although similar to a process in S18.

After S34, the processor 31 performs processing for recording inspection result information (S35). The recording calculation in S35 is performed for all the blades in S35, although similar to a process in S19.

After S35, the processor 31 performs display processing (S36). A process in S36 constitutes a display image generation section configured to generate a display image for displaying information about the detected changed area of the subject on the monitor 32. For example, processing for displaying presence or absence of existence of the blade in which an abnormal area has been found out and displaying as a list the blades in which respective abnormal areas have been found out on the monitor 32 is performed in S36. Processing for displaying an image obtained by superimposing the difference image on the visible light image on the monitor 32 is performed for the blade selected in the list of blades the respective abnormal areas of which have been found out.

As described above, according to the modification, a similar effect to the effect of the above-described embodiment is produced.

Note that, although in the above-described embodiment and modification, the hot air HA is blown to the subject to heat the subject, cool air may be blown to the subject. In the case, the above-described heating device 14 is replaced with a cooling device, and the heat generation unit 14a is replaced with a cooling unit. That is, a predetermined area of the subject is cooled, and presence or absence of an abnormal area is judged from a time series variation of a heat distribution of the cooled subject.

### Second Embodiment

Although the subject is positively heated, and the thermography image is then acquired in the above-described first embodiment, a thermography image is acquired in a process for radiating heat from the time when a subject is in a heated state, in a second embodiment.

For example, when a turbine of an electric generator enters a stopped state from an operating state, an engine starts to radiate heat from a high-temperature state. Thus, an internal temperature decreases. When the internal temperature changes, a temperature change of a blade as the subject is detected by a thermography image, to detect a minute abnormal area or the like of the subject.

Although the present embodiment will be described below, an image pickup system according to the present embodiment has a substantially identical configuration to the configuration of the image pickup system according to the first embodiment. Accordingly, description of a system configuration according to the present embodiment is omitted, and the same components as the components in the image pickup system according to the first embodiment will be described using the same reference numerals.

Also in the present embodiment, description is made with an example of inspection of a turbine blade.

In the present embodiment, for all a plurality of blades B of a rotor R, firstly, a first thermography image is acquired for all the blades B, and a second thermography image is then acquired for all the blades B. Then, difference, comparison, or the like is performed for each of the blades B.

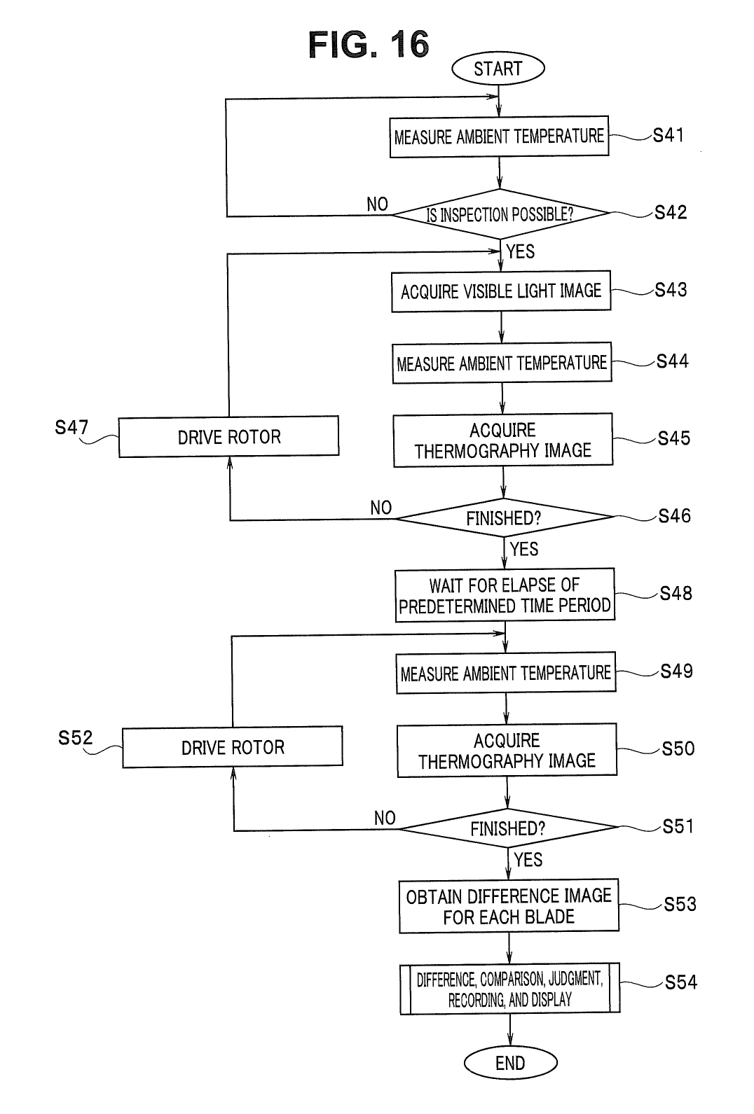

FIG. 16 is a flowchart illustrating an example of flow of processing for inspecting each of the blades B by an image pickup system performed when each of the blades B of the rotor R is inspected.

As described above, when the inspection is started, a user inserts an insertion section 11 from an access port of the rotor R, and fixes a distal end portion 11a to a position where an image of each of the blades B can be picked up. When the user operates a user interface (U/I) section 33 or an operation instrument 12a, a processor 31 performs processing illustrated in FIG. 16.

The inspection of each of the blades B of the rotor R is started from the time when an ambient temperature of the blade B has reached a predetermined temperature or less.

The predetermined temperature is a temperature at which an abnormal area can be detected from a difference in temperature distribution between the blades B when second temperature measurement is performed after a lapse of a predetermined time period since first temperature measurement was performed in inspection described below.

Accordingly, the processor 31 measures an ambient temperature of the blade B from an output signal of a thermistor 24 in the insertion section 11 (S41).

The processor 31 judges whether or not inspection is possible based on whether or not the measured temperature has reached the predetermined temperature or less (S42).

When the inspection is not possible (NO in S42), the processing returns to S41.

When the inspection becomes possible (YES in S42), the processor 31 acquires a visible light image of the blade B (S43). That is, the processor 31 acquires a visible light image of a subject by reflected light from the subject which has passed through a first observation window 16, i.e., an endoscope image by normal light.

The endoscope image is acquired for the first blade.

Subsequently to S43, the processor 31 measures an ambient temperature of the blade B (S44). The measurement of the ambient temperature of the blade B is calculated from a detection signal of a thermistor.

The processor 31 acquires a thermography image TI (S45).

The processor 31 judges whether or not image acquisition has been finished for all the blades B of the rotor R, i.e., whether or not image acquisition for the last blade B has been finished (S46).

When the image acquisition for all the blades B has not been finished (NO in S46), the processor 31 rotates the rotor R by a predetermined angle (S47). After the adjacent blade B has reached a position where an image of the blade B can be picked up using the endoscope 2 in S47, the processor 31 performs processes in S43 and subsequent steps.

When the image acquisition for all the blades B has been finished (YES in S46), the processor 31 waits until a predetermined time period t1 elapses without doing anything (S48). That is, the processor 31 measures time until the predetermined time period t1 elapses using an internal software counter.

When the predetermined time period t1 elapses, the processor 31 measures an ambient temperature of the blade B (S49).

Subsequently to S49, the processor 31 acquires a thermography image TI (S50). The thermography image is acquired for the first blade.

Subsequently to S50, the processor 31 judges whether or not the image acquisition for all the blades B of the rotor R has been finished, i.e., whether or not the image acquisition for the last blade B has been finished (SM).

When the image acquisition for all the blades B has not been finished (NO in S51), the processor 31 rotates the rotor R by a predetermined angle (S52). After the adjacent blade B has reached a position where an image of the blade B can be picked up using the endoscope 2 in S52, the processor 31 performs processes in S49 and subsequent steps.

When the image acquisition for all the blades B has been finished (YES in S51), the processor 31 generates a difference image between the thermography image obtained in S45 and the thermography image obtained in S50 for each of the blades B (S53). In S53, the difference image is generated for each of the blades B. The difference image generated in S53 is a thermography image representing a difference between two images obtained by image pickup of the subject using the thermography camera 22 at intervals of a time period including a predetermined time period t1.

Note that the generation of the difference image for each of the blades B may be performed after S50.

The processor 31 performs difference, comparison, judgment, recording, and display processing for each of the generated difference images (S54).

A reference thermography image TIREF used in the present embodiment is a thermography image representing a difference between two images obtained by image pickup of a sample as a reference of the subject using the thermography camera 22 at intervals of the time period including the predetermined time period t1.

A process in S54 includes the processes in S32 to S36 illustrated in FIG. 15 in the first embodiment. When a change in temperature distribution is a change which is a predetermined threshold value or more compared to the reference thermography image TIREF for the difference image for each of the blades B, judgment information such as a flag is recorded in a memory for the blade which includes an abnormal area or may include an abnormal area.

Accordingly, difference calculation in S54 constitutes a temperature distribution change amount detection section configured to detect a change amount in a temperature distribution of the subject. The change amount is detected based on respective pixel values for each pixel of two thermography images.

The comparison processing in S54 constitutes a changed area detection section configured to detect a changed area of the subject.

Further, the pixel values of the thermography images obtained in S45 and S50 are respectively corrected based on the ambient temperatures obtained in S44 and S49.

The reference thermography image TIREF used in the comparison processing performed in S54 is also generated using a reference sample BSP of the subject, like in the first embodiment. The reference thermography image TIREF is a reference difference image as a difference image between two images obtained by image pickup at a first time and a second time elapsed by a predetermined time period t1 or more from the first time in a process for the reference sample BSP of the subject to radiate heat. The reference difference image is previously stored in a storage region 41a in a storage device 41.

In the display processing in S54, respective numbers of the blades B each of which includes an abnormal area or may include an abnormal area may be displayed in a list format on the monitor, or a visible light image and a difference image for the blade B including an abnormal area may be displayed side by side. The display processing in S54 constitutes a display image generation section configured to generate a display image for displaying information about a detected changed area of the subject on the monitor 32.

As described above, according to the present embodiment, a similar effect to the effect of the first embodiment is produced.

A modification to the above-described second embodiment will be described below.

### Modification 2

Although the first thermography is acquired for the plurality of blades B of the rotor R, and the second thermography image is then acquired again for the plurality of blades B in the above-described second embodiment, two thermography images are acquired for each of blades B in the modification 2. For example, processing for acquiring the second thermography image after waiting for a predetermined time period since the first thermography image was acquired is performed for one of blades B, to perform processing for acquiring the two thermography images for the one blade B, and processing for acquiring similar two thermography images for the subsequent blade B is then performed.

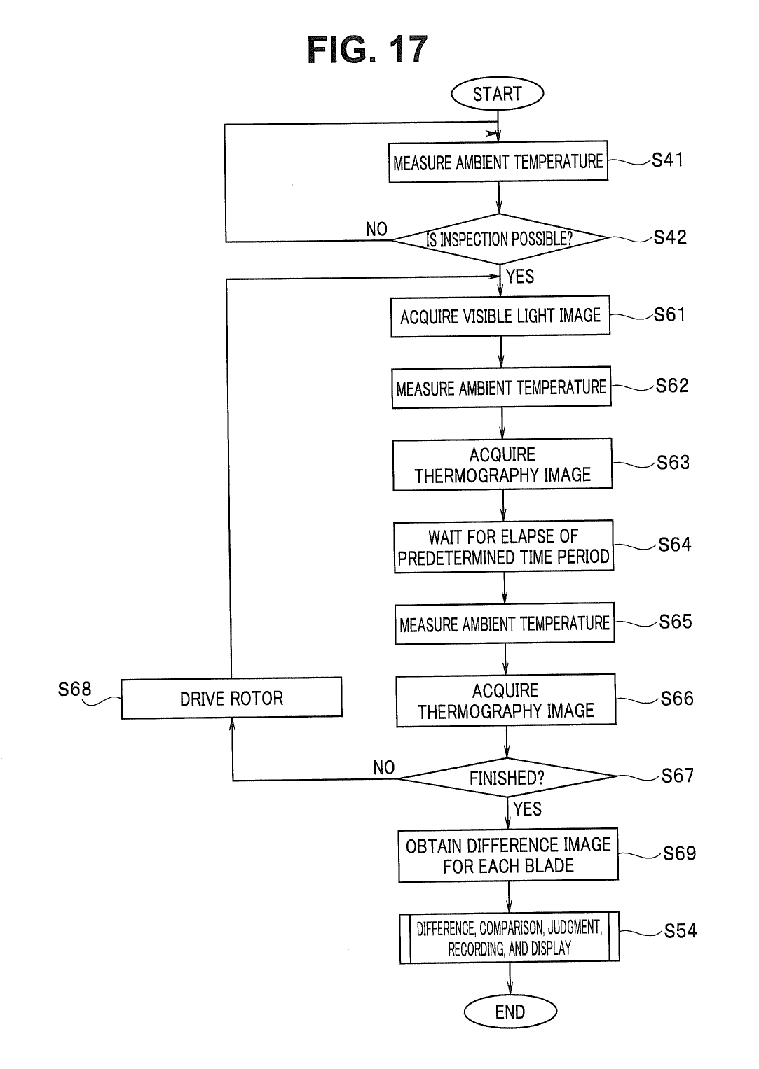

FIG. 17 is a flowchart illustrating an example of flow of processing for inspecting each of blades B by an image pickup system performed when each of blades B of a rotor R is inspected according to a modification to the present embodiment. Note that in FIG. 17, the same processes as the processes illustrated in FIG. 16 are assigned the same step numbers, and hence descriptions of the same processes are not repeated.

After a process in S42, i.e., when inspection becomes possible (YES in S42), a processor 31 acquires a visible light image of the blade B (S61).

Subsequently to S61, the processor 31 measures an ambient temperature of the blade B (S62). The measurement of the ambient temperature of the blade B is calculated from a detection signal of a thermistor.

The processor 31 acquires a thermography image TI (S63).

After S63, the processor 31 waits until a predetermined time period t2 elapses without doing anything (S64). That is, the processor 31 measures time until the predetermined time period t2 elapses by an internal software counter.

When the predetermined time period t2 elapses, the processor 31 measures an ambient temperature of the blade B (S65).

Subsequently to S65, the processor 31 acquires a thermography image TI (S66). That is, when the predetermined time period t2 has elapsed since the first thermography image was acquired in S63, a second thermography image TI is acquired.

Subsequently to S66, the processor 31 judges whether or not image acquisition for all the blades B of the rotor R has been finished, i.e., whether or not image acquisition for the last blade B has been finished (S67).

When the image acquisition for all the blades B has not been finished (NO in S67), the processor 31 rotates the rotor R by a predetermined angle (S68). In S68, after the adjacent blade B has reached a position where an image of the blade B can be picked up using an endoscope 2, the processor 31 performs processes in S61 and subsequent steps.

When the image acquisition for all the blades B has been finished (YES in S67), the processor 31 generates a difference image between the thermography image obtained in S63 and the thermography image obtained in S66 for each of the blades B (S69). In S69, the difference image is generated for each of the blades B.

Note that the generation of the difference image for each of the blades B may be performed after S66.

The processor 31 performs difference, comparison, judgment, recording, and display processing for each of the generated difference images (S54).

As described above, according to the modification, a similar effect to the effect of the above-described second embodiment is also produced.

Note that, although the thermography image is acquired in a process for the subject to be cooled after stopping operating in the above-described second embodiment and modification, a thermography image in a process for the subject to be warmed by heat absorption may be acquired after the entire subject is cooled by being placed in a refrigerator or the like. That is, presence or absence of an abnormal area is judged from a time series change of a heat distribution of the subject in the process for the subject to be warmed.

As described above, according to each of the above-described embodiments and modifications, the image pickup apparatus, the image pickup system, and the image pickup method capable of detecting the abnormal area which cannot be found out or is not easily found out in the visible light image can be provided.

Note that, although the information about the difference image is superimposed and displayed, for example, in the display processing in each of the above-described embodiments and modifications, information about a detected changed area may be displayed on the monitor 32 by image processing such as pan/tilt for moving the changed area to a center of the monitor 32 or a bending operation of the insertion section 11.

A modification common to both the above-described two embodiments and two modifications will be described below.

### Modification 3

Although hot air is blown to the subject in each of the above-described embodiments and modifications, a heated member (or a cooled member) may be brought into contact with the subject to heat (or cool) of the subject.

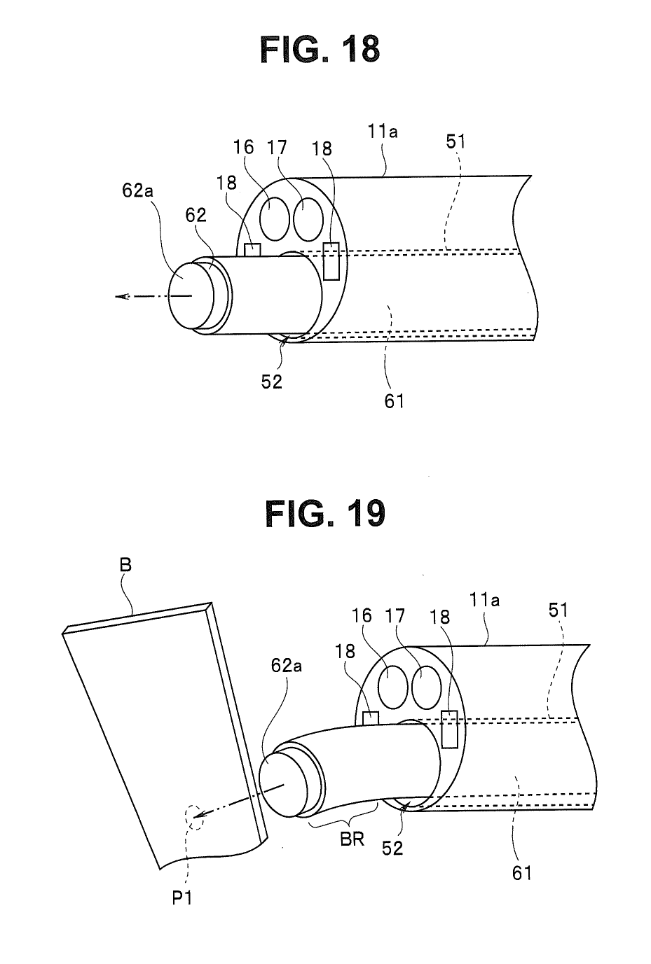

FIG. 18 is a perspective view illustrating a configuration of an insertion section of an endoscope including a heating member. An insertion section 11 includes a treatment instrument insertion channel 51. The treatment instrument insertion channel 51 is formed parallel to a central axis of the insertion section 11. A distal end portion of the treatment instrument insertion channel 51 is an opening portion 52, and a proximal end portion (not illustrated) communicates with a treatment instrument insertion opening (not illustrated) formed in close proximity to an operation section 12, for example.

A heating treatment instrument 61 is an elongated probe, and includes a heating element 62 in its distal end portion. A distal end of the heating element 62 has a distal end surface 62a that is a plane. The heating element 62 is a heater by a resistor or a Peltier element, and generates heat by causing a predetermined current to flow. That is, the heating treatment instrument 61 includes the heating element 62 configured to heat a subject in contact with a part of the subject.

Note that the heating element 62 may be an ultrasound transducer. When the ultrasound transducer ultrasonically vibrates, the subject can be heated with a frictional heat.

Accordingly, a user can heat a predetermined position P1 of the subject by inserting the heating treatment instrument 61 into the treatment instrument insertion channel 51 in the insertion section 11 and causing the heating treatment instrument 61 to protrude from the opening portion 52 and contact the subject.

Note that a bending habit may be formed in a distal end portion of the heating treatment instrument 61. FIG. 19 is a perspective view of the heating treatment instrument 61 having a bending habit in the distal end portion.

Since the distal end portion BR of the heating treatment instrument 61 has a bending habit, the heating treatment instrument 61 is bent in a predetermined direction when it protrudes from the opening portion 52. Accordingly, the user can easily heat the predetermined position P1 of the subject such as a blade B with the distal end surface 62a opposing a surface of the subject when the surface of the subject has an angle to a surface perpendicular to an insertion direction of the insertion section 11.

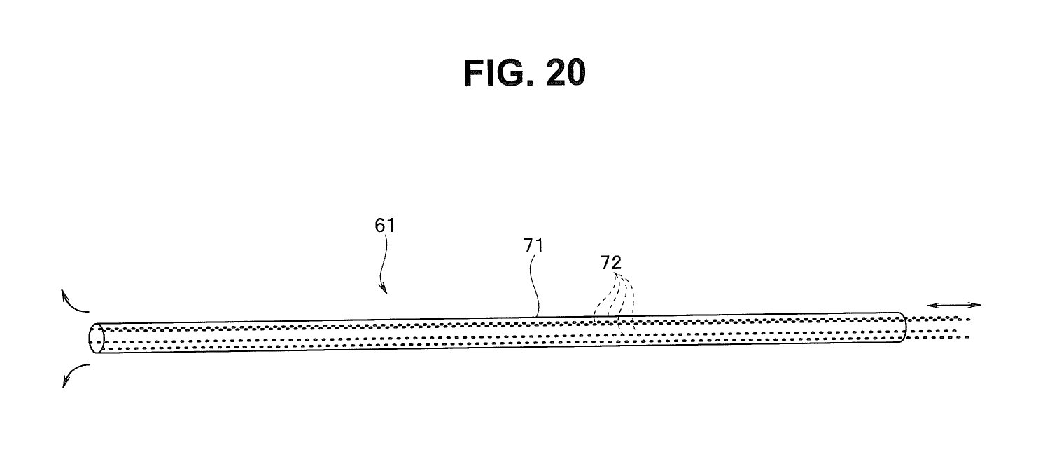

Note that the heating treatment instrument 61 may have a bending mechanism without having a bending habit in the distal end portion. FIG. 20 is a diagram for describing the bending mechanism of the heating treatment instrument.

The heating treatment instrument 61 includes a probe member 71 a distal end portion of which can be bent. The elongated probe member 71 having flexibility is a cylindrical member, and four bending wires 72 are inserted into the probe member 71. Respective distal end portions of the bending wires 72 are fixed to a distal end of the probe member 71 while being equally spaced around a central axis of the probe member 71.

Accordingly, when the user performs a bending operation for pulling at least one of the four bending wires 72 and relaxing the other bending wires, the user can bend the distal end portion of the heating treatment instrument 61 in a desired direction. The heating treatment instrument including such a bending portion can heat the subject with the distal end surface 62a of the heating element 62 opposing the surface of the subject.

Further, note that, although the heating treatment instrument 61 is inserted into the treatment instrument insertion channel 51 to heat the subject in the above-described example, a cooling element is provided in a distal end portion of a probe instead of the heating element 62 when the subject is cooled. The cooling element is a Peltier element, for example.

Therefore, according to the modification 3, a similar effect to the effect produced by each of the above-described embodiments and modifications is also produced.

### Modification 4

Although the subject is heated or cooled using hot air, cold air, a heating treatment instrument, or a cooling treatment instrument for the subject in each of the above-described embodiments and modifications, a subject may be cooled by ambient air using cooling by suction.

When a suction device is connected to a proximal end portion of a treatment instrument insertion channel 51 in an insertion section 11 illustrated in FIG. 18, and is driven with an opening portion 52 positioned in substantially close proximity to a predetermined position P1 of a subject, a portion of the subject in close proximity to the opening portion 52 is cooled by air movement at the time when ambient air is sucked in from the opening portion 52.

Accordingly, the user can cool the predetermined position P1 of the subject by bringing a distal end portion 11a close to the predetermined position P1 of the subject to suck in air from the opening portion 52.

Therefore, according to the modification 4, a similar effect to the effect produced by each of the above-described embodiments and modifications is also produced.

### Modification 5

Although the processor 31 in the main body section 13 outputs the control signal to the turning device 44 to rotate the rotor R within the turbine to inspect the plurality of blades B in each of the above-described embodiments and modifications, the user may manually operate the turning device 44.

Furthermore, when the inspection for the one blade is finished without the rotor R being rotated, the user may move the insertion section 11 to proximity of a subsequent subject.

### Modification 6

Although the thermography image is corrected based on the ambient temperature in each of the above-described embodiments and modifications, a heating amount or a cooling amount for heating or cooling the subject depending on the ambient temperature may be adjusted such that a similar heat distribution to the heat distribution obtained when the sample image has been obtained is obtained.

### Modification 7

Although each of the pixel values of the difference image and the threshold value are compared with each other to judge the presence or absence of the abnormal area in the judgment processing in each of the above-described embodiments and modifications, the type of abnormal area may be estimated and judged based on a shape, a position, or the like of the region RS of the pixel having the pixel value which is the threshold value TH or more in the difference image.

The present invention is not limited to the above-described embodiments and modifications, but various changes, alterations, or the like may be made without departing from the gist of the present invention.

What is claimed is:

1. An image pickup apparatus comprising:
a thermography camera configured to pick up an image of an infrared light band; and
a processor including a hardware,
the processor being configured to:
detect, from a difference in a temperature distribution between at least one thermography image for at least one subject obtained by image pickup of the at least one subject using the thermography camera and a reference thermography image obtained by image pickup of the at least one subject using the thermography camera, a change amount in the temperature distribution of the at least one subject;
detect a changed area of the at least one subject where the detected change amount is a predetermined value or more; and
generate a display image for displaying information about the detected changed area on a display device.

2. The image pickup apparatus according to claim 1, wherein the processor detects the change amount based on respective pixel values for each pixel of the at least one thermography image and the reference thermography image.

3. The image pickup apparatus according to claim 1, further comprising a visible light camera configured to pick up an image of a visible light band to acquire a visible light image,
wherein the processor superimposes the information about the changed area on the visible light image obtained by the image pickup using the visible light camera.

4. The image pickup apparatus according to claim 1, wherein the processor superimposes the information about the changed area on the at least one thermography image.

5. The image pickup apparatus according to claim 1, wherein the information is a difference image between the at least one thermography image and the reference thermography image.

6. The image pickup apparatus according to claim 1, further comprising a heating/cooling device configured to heat or cool the at least one subject, wherein
the at least one thermography image is an image obtained by image pickup using the thermography camera after the heating/cooling device heats or cools a part of the at least one subject for a first predetermined time period, and
the reference thermography image is an image obtained by image pickup using the thermography camera after the heating/cooling device heats or cools a part of a sample as a reference of the at least one subject for the first predetermined time period.

7. The image pickup apparatus according to claim 6, wherein the heating/cooling device blows hot air or cold air to the at least one subject to heat or cool a part of the at least one subject.

8. The image pickup apparatus according to claim 6, wherein the heating/cooling device includes a heating or a cooling element configured to heat or cool the at least one subject in contact with the part of the at least one subject.

9. The image pickup apparatus according to claim 1, further comprising a storage device configured to store the reference thermography image,
wherein the processor reads out the reference thermography image from the storage device, to detect the change amount in the temperature distribution of the at least one subject.

10. The image pickup apparatus according to claim 9, wherein the reference thermography image is an image obtained by image pickup of a sample as a reference of the at least one subject.

11. The image pickup apparatus according to claim 1, wherein the at least one subject includes a plurality of subjects,
wherein the processor determines the reference thermography image based on a plurality of thermography images respectively obtained by image pickup of the plurality of subjects using the thermography camera, and
the processor detects a change amount in a temperature distribution of each of the subjects based on a corresponding one of the plurality of thermography images and the determined reference thermography image.

12. The image pickup apparatus according to claim 1, wherein the at least one thermography image is a difference image between two images obtained by image pickup of the at least one subject using the thermography camera at intervals of a second predetermined time period.

13. The image pickup apparatus according to claim 12, wherein the reference thermography image is a difference image between two images obtained by image pickup of a sample as a reference of the at least one subject using the thermography camera at intervals of the second predetermined time period.

14. The image pickup apparatus according to claim 1, wherein
the at least one subject includes a plurality of subjects, the subjects having an identical shape, and
the processor detects the change amount in the temperature distribution of each of the subjects.

15. An image pickup system comprising:
an endoscope including an insertion section;
a thermography camera provided in a distal end portion of the insertion section and configured to pick up an image of an infrared light band; and
a processor including a hardware,
the processor being configured to:
detect, from a difference in a temperature distribution between a thermography image for a subject obtained by image pickup of the subject using the thermography camera and a reference thermography image obtained by image pickup of the subject using the thermography camera, a change amount in the temperature distribution of the subject;
detect a changed area of the subject where the detected change amount is a predetermined value or more; and
generate a display image for displaying information about the detected changed area on a display device.

16. The image pickup system according to claim 15, wherein
the insertion section includes a visible light camera configured to pick up an image of a visible light band to acquire a visible light image in the distal end portion, and
the processor superimposes the information about the abnormal area on the visible light image or the thermography image obtained by the image pickup using the visible light camera.

17. An image pickup method comprising:
picking up an image of a subject to acquire a thermography image for the subject using a thermography camera configured to pick up an image of an infrared light band;
detecting, from a difference in a temperature distribution between the thermography image and a reference thermography image obtained by image pickup of the subject using the thermography camera, a change amount in the temperature distribution of the subject;
detecting a changed area of the subject where the detected change amount is a predetermined value or more; and
displaying information about the detected changed area on a display device.
